(19) United States
(12) Patent Application Publication
(10) Pub. No.: US 2020/0069928 A1
(43) Pub. Date: Mar. 5, 2020

(54) TRIGGERING OF PAYLOAD RELEASE FROM MINIATURIZED DEVICES

(71) Applicant: BIONAUT LABS LTD., Herzliya (IL)

(72) Inventors: Michael SHPIGELMACHER, Los Angeles, CA (US); Alex KISELYOV, San Diego, CA (US)

(73) Assignee: BIONAUT LABS LTD., Herzliya (IL)

(21) Appl. No.: 16/615,654

(22) PCT Filed: May 3, 2018
(86) PCT No.: PCT/US18/30953

§ 371 Date: Nov. 21, 2019

Related U.S. Application Data
(60) Provisional application No. 62/512,091, filed on May 29, 2017.

(51) Int. Cl. A61M 37/00

(52) U.S. Cl. CPC A61M 37/0092; A61M 2205/0238; A61M 2205/0288; A61M 2207/00

(57) ABSTRACT

A carrier device and methods of use are described. The device and methods are directed toward implanting in biological tissue and optionally for propelling in a biological tissue and for releasing a medical payload in a biological tissue according to remote trigger. The carrier device includes at least one element sensitive to the external stimuli. When external stimuli are sent through the tissue, the responsive element provides release of the functional material. In some embodiments, payload release can be started, stopped, and restarted at a later time or place. Individual carrier devices can be selectively triggered by implementing different elements in the devices, each element is sensitive to different stimuli. In addition to payload release, devices of this invention are equipped with a propelling element, the propelling element is responsive to external stimuli that enables propulsion and navigation of the device.

### CROSS REFERENCE TO RELATED APPLICATIONS

This application claims benefit of U.S. Provisional Patent Application Ser. No. 62/512,091, filed May 29, 2017, the priority date of which is hereby claimed.

### BACKGROUND

Ultrasound (US)—based methods currently exist for remotely triggering release of a medical payload, such as drugs and diagnostic aids, from particles or devices implanted in a living tissue. US Remotely-triggered payload release from particles or implantable devices have been researched in the past. The purpose of such methods is to generate an external trigger for payload release (drug or diagnostics) from a carrier (e.g., particle or implantable device) housing such a payload in a living tissue. Remotely-triggered payload release is desirable in supporting specific clinical goal, such as:

Release of a medical payload only when the carrier particle is in the right location for treatment (e.g., tumor);

Release of a medical payload only when the moment is right (e.g., in the middle of a clinical procedure); or

Release of a medical payload in a time-dependent or stop-and-go manner to treat predetermined area(s) for a predetermined time.

Existing US-based trigger methods rely on a variety of effects, including:

Thermal/mechanical effect based on cavitation (leading to localized heating due to vibration and increased speed of diffusion and/or changes in localized chemical

Mechanical degrading/rupturing of carrier leading to payload release;

Shape change of the carrier or an integrated part thereof; or

Change to the characteristics of the surrounding biological tissue into which the payload is being released (e.g., sonoporation), resulting in improved payload diffusion/absorption through tissue.

A common drawback of these methods is that each method supports only a subset of the typical technical features desired from a clinical standpoint. These features for an ultrasound-based remote trigger system for clinical payload release include:

Customizable tissue penetration depth (10 cm or greater) to be able to trigger payload release in deeply situated tissue. For instance, release methods in the >7 MHz (diagnostic US) range are typically limited to less than 10 cm penetration.

Customizable frequency range (KHz-MHz range) to offer compatibility with existing medical ultrasound equipment and to minimize invasiveness to tissue. For example, cavitation-based methods are typically most effective in the KHz range using high intensity focused ultrasound (HIFU), while polymer degradation-based methods are more effective in the MHz range (diagnostic US);

Support for gradual payload release over a controllable time period, or an on-off switchable release functionality (rather than a single release pulse). For example, methods relying on degradation of a uniform polymer encasing the payload are by design irreversible and do not have gradual release functionality; and

Individual control of multiple payload carriers in a single tissue volume unit (e.g., releasing payload selectively from only a single particle out of many located within the same organ). None of the existing methods offer this functionality.

It would therefore be desirable to have implantable devices and methods thereof, which overcome the above restrictions of the current capabilities This goal is attained by embodiments of the present invention.

### SUMMARY

According to various embodiments of the present invention, there is provided an implantable payload carrier device with at least one US sensitive element to implement:

customizable tissue penetration depths;

support for customizable US frequency ranges;

gradual and on/off switchable payload release capabilities;

individual control of multiple payload carriers in the same tissue region; and

methods for use of the above devices.

The significance of these features is as follows:

1. Customizable tissue penetration depth (10 cm or greater), to be able to trigger payload release in deeply situated tissue. For instance, release methods in the >7 MHz (diagnostic US) range are typically limited to less than 10 cm penetration.

2. Customizable frequency range (KHz-MHz range), to offer compatibility with existing medical ultrasound equipment and to minimize invasiveness to tissue. For example, cavitation-based methods are typically most effective in the KHz range using HIFU, while polymer degradation-based methods are more effective in the MHz range (diagnostic US).

3. Support of gradual payload release over a controllable time period, or an on-off switchable release functionality (rather than a single release pulse). For example, methods relying on degradation of a uniform polymer encasing the payload are by design irreversible and do not have the gradual release functionality.

4. Individual control of multiple payload carriers in a single tissue volume unit (e.g., releasing payload selectively from only a single particle out of many located within the same organ). None of the existing methods offer this functionality

Certain embodiments of the present invention rely on ultrasound (US) for remote triggering and navigation of carriers implanted in living tissue. Other embodiments combine ultrasound with other external physical stimuli, non-limiting examples of which include: electromagnetic fields, phenomena, and effects; and thermodynamic phenomena and effects, including both temperature and pressure effects.

The terms “carrier device” and “carrier” herein denote any object that is implantable in biological tissue, and is capable of carrying and releasing a medical payload into the tissue. In some embodiments, the term “device” or the term “particle” are used to describe the carrier or the carrier device. The term “medical payload”, or equivalently the term “payload” used in a medical context is understood herein to include any substance or material of a medically-therapeutic or diagnostic nature. In certain embodiments, the medical payload or payload is equivalent to a “functional material” wherein the function is related to or directed toward treatment or for diagnostic purposes. The term “device” (with reference to a carrier) herein denotes a carrier which is fabricated by manufacturing techniques, including, but not limited to, lithography, thin-film technologies, deposition technologies, etching, coating, molding, self-assembly, chemical synthesis and the like. The term “particle” in some embodiments of this invention is noted with reference to a carrier device.

In various embodiments of the present invention, carrier devices are miniaturized for implantation in biological tissues. The term “miniaturized” (with reference to a carrier) herein denotes a carrier of small size, including, but not limited to: carriers of millimeter to centimeter scale; carriers of micrometer (“micron”) scale, referred to as “carrier micro-devices”; carriers of nanometer scale referred to as “carrier nano-devices”. Not only are the carriers themselves of the size scales as indicated above, but the carriers' individual components are also of comparable scale. It is to be noted that certain carrier dimensions can be of different scales, e.g., a carrier may have one dimension in the nanometer range and another dimension in the micrometer range. All such miniatured devices are included in embodiments of this invention.

In one embodiment, this invention provides a carrier device for implanting in a biological tissue for release of a functional material in said tissue or in another tissue, the carrier device comprising:

a structure comprising a propelling component;

a functional material attached directly or through a linker to said structure; and

optionally a coating, said coating at least partially covers said structure and at least partially covers said functional material attached to said structure.

In one embodiment, the propelling component is a magnetic component. In one embodiment, the propelling component, said functional material, said coating or a combination thereof are responsive to external stimuli. In one embodiment, stimuli are selected from US, magnetic, electric, electromagnetic, electromagnetic radiation or a combination thereof. In one embodiment, the application of said stimuli to said propelling component propels said device. In one embodiment, in devices of this invention:

said functional material detaches from said structure in response to said external stimuli; or

said coating ruptures or becomes perforated in response to said external stimuli; or

a combination thereof.

In one embodiment, the external stimulus is US. In one embodiment, the external stimuli comprise magnetic stimuli for propelling the propelling component and US stimuli for releasing the functional material from said device or from components thereof.

In one embodiment the structure is at least partially porous. In one embodiment the average pore size of said porous structure ranges between 10nm-1000 nm. In one embodiment, the functional material is or comprises an organic compound, a polymer, a composite or a combination thereof. In one embodiment the coating comprising a polymer, a composite or a combination thereof. In one embodiment, the structure is a microstructure, a nanostructure or a combination thereof.

In one embodiment, this invention provides a system comprising:

a device as disclosed herein; and

wherein the remote unit is configured to apply external stimuli to said device.

In one embodiment, the external stimuli comprise US. In one embodiment, the external stimuli comprise US and magnetic stimuli.

In one embodiment, the functional material changes its shape or topology, or detaches from said structure in response to said external stimuli; or said coating ruptures or becomes perforated in response to said external stimuli; or a combination thereof.

In one embodiment, this invention provides a method for operating a device, said method comprising:

providing a carrier device comprising:
a structure comprising a propelling component;
a functional material attached directly or through a linker to said structure;
optionally a coating, said coating at least partially covers said structure and at least partially covers said functional material attached to said structure; and

applying external stimuli to said device.

In one embodiment, the coating, said functional material or a combination thereof are responsive to said external stimuli. In one embodiment, the stimulus is US. In one embodiment, the stimuli comprise magnetic stimuli for propelling the propelling component and US stimuli for releasing the functional material from said device.

In some embodiments, in methods of this invention:

the functional material detaches from said structure in response to said external stimuli; or

the coating ruptures or becomes perforated or assumes on open position in response to said external stimuli; or

a combination thereof.

In one embodiment the functional material is or comprises an organic compound, a polymer, a composite or a combination thereof. In one embodiment the coating comprising a polymer, a composite or a combination thereof.

In one embodiment the structure is a microstructure, a nanostructure or a combination thereof. In one embodiment the propelling component comprises a magnetic component.

In one embodiment, this invention provides a method of producing the device of this invention, said method comprising:

providing or constructing a propelling structure, said structure comprises a propelling element;

binding a functional material to said structure; and

optionally coating at least partially said structure or optionally coating at least partially said functional material or a combination thereof.

In one embodiment, this invention provides a method of treating a subject, said method comprises:

inserting a device described herein into said subject;

applying external stimuli to said device.

In one embodiment, inserting the device comprises inserting the device into a certain tissue within said subject.

In one embodiment the external stimuli comprise:

magnetic/electric or electromagnetic stimuli to propel the device to a defined location within the subject; or

US stimuli to detach said functional material from said structure and/or to rupture or perforate said coating; or

a combination thereof.

In one embodiment following application of said external stimuli, said functional material interacts with said tissue or with component(s) of/in said tissue. In one embodiment, the interaction results in a therapeutic effect, a diagnostic effect or a combination thereof. In one embodiment. the method further comprises imaging the location of said device within said subject. In one embodiment, the propelling component is a magnetic component.

### BRIEF DESCRIPTION OF THE DRAWINGS

The subject matter disclosed may best be understood by reference to the following detailed description when read with the accompanying drawings in which:

FIG. 1 illustrates representative examples of ultrasound-sensitive (US) composite particles. FIG. 1.1 is an example of an ultrasound (US-sensitive polymer coat; FIG. 1.2 is a representative example for synthesis of the US sensitive particle with magnetic core and mesoporous loading components; FIG. 1.3 is a representative example for a particle with US-sensitive chelating surface; and FIG. 1.4 is a representative example for temperature-sensitive (ex. shape-memory, expand/collapse, degradable) coating and US heated core metal/composite heated by US.

FIG. 2 is a representative particle with an etched US-sensitive chelating surface.

FIG. 3 shows examples for US-sensitive chemical bonds that yield polar moieties upon cleavage.

For simplicity and clarity of illustration, elements shown in the figures are not necessarily drawn to scale, and the dimensions of some elements may be exaggerated relative to other elements. In addition, reference numerals may be repeated among the figures to indicate corresponding or analogous elements.

### DETAILED DESCRIPTION

Various embodiments of the present invention provide a carrier device containing a functional material which is released from the carrier upon demand. The term “functional material” includes any substance, compound or material of a medically-therapeutic or diagnostic nature. The functional material is released from the carrier when external stimuli are applied. The external stimuli can be electric, magnetic, electro-magnetic, electromagnetic radiation, ultrasound, or a combination thereof. In some embodiments, the functional material is provided attached to another material or comprised within another material. In some embodiments, the functional material is part of a composition. According to this aspect embodiments that refer to the functional material may also refer to a larger entity/composition that comprises the functional material.

In some embodiments of the present invention, the carrier device and its component parts are miniaturized. The device and/or the structures included in the device have at least one dimension at the microscale, the nanoscale or a combination thereof. According to some embodiments, the diameter or actual length of the overall device is selected from: between 100 and 5,000 micrometers, between 10 and 100 micrometers, between 1 and 10 micrometers, between 200 and 1,000 nanometers, and any combination thereof. According to some embodiments, the diameter or actual length of the overall device is from 200 nanometers up to 5,000 micrometers.

In some embodiments of the present invention, a carrier device comprises a shape selected from elongated, axisymmetric, centrosymmetric, chiral, random and any combination thereof.

Following are detailed descriptions of some non-limiting embodiments, with reference to drawings thereof.

In one embodiment, this invention provides a method to manufacture payload carriers wherein the payload can be released based on an external ultrasound trigger/stimulus at a predefined frequency X, while potentially supporting remote-controlled motion of the carrier using an externally applied electromagnetic field. In one embodiment, the payload is or comprises functional material. In one embodiment, the particle is at halt when releasing the payload. In other embodiments, the particle is in motion while releasing the payload.

One design of the ultrasound-responsive micro/nanoparticle includes the following:

1. A combination of:
a) a magnetic component (or other propelling component) for navigation and/or ultrasound-induced heating;
b) a loading core for absorption/adsorption or covalent attachment of a payload (i.e. a functional material); and optionally
c) ultrasound-sensitive insulating coating that is responsive to a specific ultrasound frequency or frequency range;

2. The coating secures the payload and is specifically designed to be removed at will to release the payload at the designated location, tissue, or organ.

3.A representative coating design may include an ultrasound-sensitive polymer film of varying thickness, single or multiple polymer layers, prefabricated polymer etching namely adding pre-determine tension pattern or irregularities including ridges, valleys; polymer comprising embedded micro-defects, as well as polymer comprising oligomers of same or various length, and polymers of any suitable chemical composition;

4. Importantly, the polymer and its components are selected to be non-toxic or rapidly metabolized to yield non-toxic fragments in order to minimize local side effects;

5. The ultrasound-sensitive coating is responsive to a specific ultrasound frequency via (a) ‘low frequency’ (10-100 KHz range) ultrasound to induce local cavitation followed by cavitation-induced removal of the coating, or (b) ‘diagnostic frequency’ (0.5-12 GHz range) ultrasound to induce direct polymer decomposition and/or ultrasound-based heating of the magnetic/mesoporous core followed by decomposition of the polymer coat;

6. Removal of the polymer coat exposes the payload to the surrounding media; this step combined with the ultrasound treatment at either low or high frequency triggers payload desorption or a covalent bond cleavage to release the payload at a specified location;

7. The particle (carrier device) can be subsequently removed and collected using a specialized magnetic collector, catheter or magnetized needle.

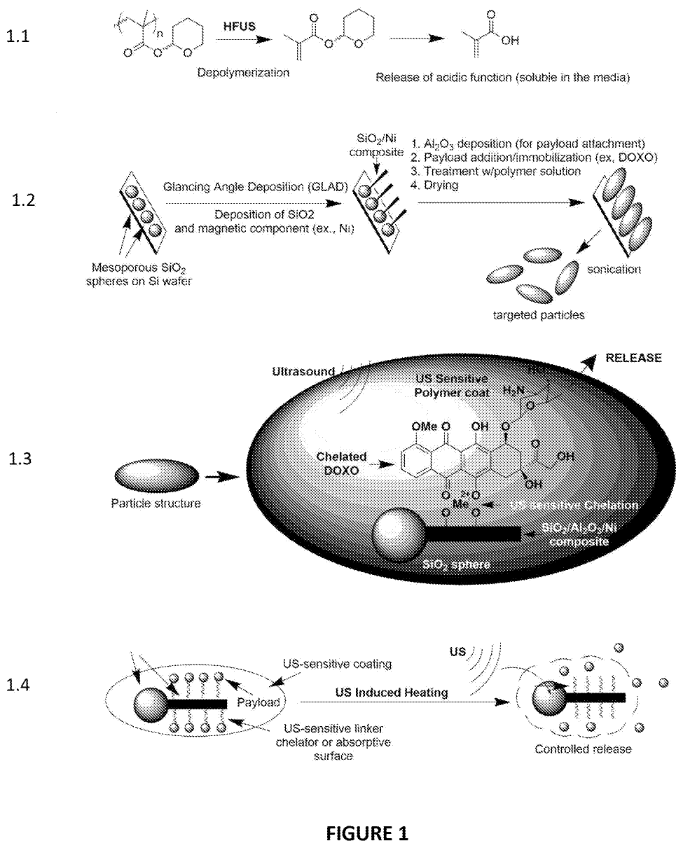

Specific details of different embodiments of this basic design are summarized below and in FIGS. 1-3.

For various applications, it may be beneficial to manufacture payload carriers (e.g., micro/nano particles) whose motion can be remotely controlled using an externally applied electromagnetic field. An example of such particles is described in U.S. Pat. No. 8,768,501, whose disclosure is incorporated herein by reference in its entirety. Such exemplified particles are magnetically-actuated propellers (MAPs). The propellers are structures with typical feature sizes in the range of 20 nm up to 100 microns in one spatial dimension. The MAPs can be produced in large numbers from nano-structures surfaces in one embodiment. The MAPs are propelled and controlled by magnetic fields. The MAPs form is a screw-like form. The screw-like MAPs are rotated and driven by a rotating magnetic field. Rotation of the MAPs around their long axis, propels them forward. A method of design for payload carriers is described below, which support such functionality, while also supporting the features summarized above pertaining to remote controlled payload release based on an ultrasound signal.

In one embodiment as shown in FIG. 1, the specific composition of the particles includes:

i. A ferro/paramagnetic component (implemented for example in the Figure as a SiO₂/Ni rod) incorporated to support particle navigation, imaging, therapeutic-diagnostics (theranostics), modulation of surface properties, for example by heating with ultrasound; the magnetic component could be incorporated into a particle via a variety of methods including multiple physical vapor deposition techniques, laser direct writing, electrodeposition, solvothermal methods, sol-gel, structured-media syntheses (such as the GLAD protocol shown in FIG. 1.2);

ii. A mesoporous component or an alternative with high loading capacity as exemplified by porous composites with cavities (e.g., pores) <20 nm or <100 nm and/or ‘gating’ material, i.e. material that can undergo pore open-close transformation via changing conformation or chemical decomposition triggered by external stimuli (e.g. inclusion of doxorubicin into a β-cyclodextrin cavity), chelating/complexing molecules (FIG. 1.3);

iii. An ultrasound (US)-sensitive coating (specific polymer film that changes topology, physical or chemical integrity reversibly (shape-memory) or irreversibly via chemical degradation or depolymerization (as exemplified by 2-tetrahydropyranyl methacrylate, induced temperature gradient on the surface) and could be further fabricated to be mono/poly-layered, etched/patterned or contain shape-memory based polymer(s) for controlled release of payload (FIGS. 1.1, 1.3, 1.4); and

iv. the particle design could be further extended to enhance imaging via conventional techniques as exemplified by ultrasound or radiography/fluorography; specifically, a ferromagnetic/metal component may contain specific image enhancing element(s) or alloy(s) as exemplified by but not limited to Nb, Zr, Ta and other rare earth metals.

In some embodiments, the magnetic component comprises a ferromagnetic or paramagnetic material. The magnetic component can be a particle/structure made of a ferro/para-magnetic material, or it can be made of a non-ferro/non-para magnetic material that is coated by a ferro/para magnetic coating layer. The ferro/para magnetic component may comprise a ferro/para magnetic portion and a non-ferro/non-para-magnetic portion attached to each other.

A particle/component/structure itself is at least partially ferromagnetic or paramagnetic in some embodiments. In some embodiments, a ferro/para-magnetic coating layer on a non-ferro/non-para magnetic material coats at least a portion of the non-magnetic material, or coats the entire exposed surface of the non-magnetic material (except for anchor points in some embodiments). One design that is applicable to embodiments of the invention is a design where the ferromagnetic or paramagnetic particles/components are partially coated by a non-magnetic material.

In embodiments where the ferromagnetic or paramagnetic particles are partially coated or are coating or are in contact with/by a non-magnetic material, non-limiting examples of such non-magnetic material include diamagnetic dielectric materials (SiO₂, alumina), diamagnetic metals (Cu, Ag, Au) and diamagnetic organic coating (organic polymers, small molecules, a chiral compound etc.).

In some embodiments, the ferromagnetic portion or paramagnetic portion is or comprises any ferromagnetic or paramagnetic substrate known in the art. In some embodiments the ferromagnetic portion comprises Co, Fe, Ni, Gd, Tb, Dy, Eu, oxides thereof, alloys thereof or mixtures thereof. In other embodiments the paramagnetic portion comprises magnetic doped semiconductors.

For example, the mesoporous component may include any of the following materials: silicon oxide (silica), zirconium oxide, titanium dioxide, niobium oxide, aluminum-based spinel, carbon. A specific composite materials combining Si/Al oxides and ferro/paramagnetic components could provide for an i) improved loading capacity; ii) better encapsulation of therapeutics in the pores; iii) regulate dynamic and kinetic pore size (e.g., custom manufacturing or expansion/shrinking via local heating with high frequency ultrasound (HFUS)); iv) potential to cap mesoporous surface via ‘protective’ HFUS-sensitive coating; v) immobilized specific gated molecules (e.g., β-cyclodextrins or dextran derivatives) with embedded therapeutic load that could be released via application of HFUS; vi) chelating/complexing surface (e.g., polycarboxylic or polyamine modification of Al₂O₃) that could coordinate specific therapeutic agents exemplified but not limited to cisplatin CDDPt or doxorubicin, FIGS. 1.2/1.3).

Mesoporous refers to a material comprising small pores usually in the nm range. However, porous materials of this invention include in some embodiments, porous, nanoporous, microporous, microporous materials with any size/size distribution of pores that fits certain embodiments of the invention. Any material with uniform size distribution of pores or with uniform pore-size range or with pores of different size ranges is contemplated.

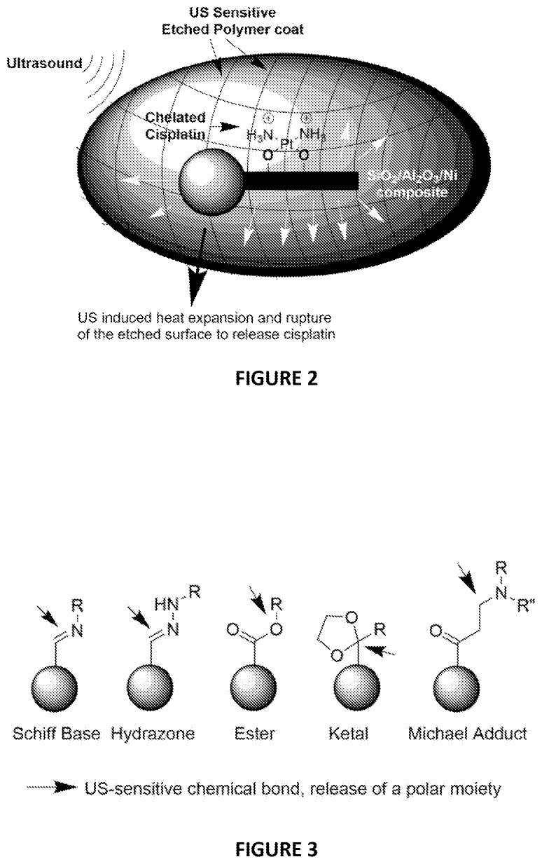

Synthesis: Specific examples of HFUS-sensitive polymers include but are not limited to PDMS or 2-tetrahydropyranyl methacrylate (THPMA) (FIG. 1.1). In a representative example, a composite particle containing ferro/paramagnetic and mesoporous Al/Si oxide components prepared via any of the physical vapor deposition (PVD) techniques (e.g., GLAD) is loaded with doxorubicin followed by treatment with saturated solution of THPMA in a polar aprotic solvent (e.g., acetonitrile or DMF) followed by drying and curing to produce a particle coated with hydrophobic polymer. HFUS treatment of the particle cleaves the acetal groups to release free carboxylic acid moieties and thus generates a hydrophilic product that is soluble in the reaction milieu ex vivo or in the tissue in vivo to expose the mesoporous surface and to release the payload. Importantly, the chemical conversion could be finetuned to support either immediate or gradual payload release by i) applying multiple lower frequency US pulses (slow release in the range of 10-100 KHz) or HFUS (fast ‘digital’ release in the range of 0.5-12 MHz); ii) varying US treatment time; iii) applying polymer layering technique or block (co)polymerization; iv) designing a patterned protective polymer surface (e.g., etched surface containing pre-set US-sensitive defects facilitating polymer film decomposition, as shown in FIG. 2) that could be gradually or immediately ruptured by US; or v) altering ferromagnetic composition/geometry to support a more efficient local temperature gradient induced by US/HFUS due to different heat conductivity by ferromagnetic materials vs. other materials. Additional chemical groups that can be incorporated into a (co)polymer in order to respond to the US stimuli and release the entrapped payload include but are not limited to Schiff bases, hydrazones, ketals, esters, Michael adducts (FIG. 3).

In a representative embodiment of this invention, particle (carrier device structure) sizes could vary between 20 nm and 1 mm. In some embodiments, the devices are in the micrometer range. In some embodiments, the devices are in the nanometer range. Within a certain range means that the largest measured dimension of the device is within that range. Devices within the millimeter range are also part of this invention. Microdevices of this invention may possess dimensions in both the nanometer and in the micrometer range Millimeter range devices may possess dimensions in the mm, μm, nm range or any combination thereof. Sizes (or largest dimension size of devices of this invention range between 20 nm and 100 nm, between 10 nm and 10 mm, between 20 nm and 1 mm, between 10 nm and 1 micron (micron=μm), between 10 nm and 10 microns, between 20 nm and 100 microns, between 1 micron to 10 microns, between 10 microns and 100 microns, between 100 microns and 1 mm, between 1 mm and 10 mm, between 1 micron and 5 mm, between 10 nm and 1 mm, between 100 nm and 1 micron, between 100 nm and 10 micron, between 100 nm and 100 micron, between 100 nm and 1000 microns. Compositions comprising particles of different sizes, different size ranges and any combination of particles of various/different sizes is included in embodiments of this invention.

In a representative embodiment of this invention, particle (carrier device structure) sizes could vary between 20 nm and 1 mm and exhibit a variety of geometries specifically selected to enhance their active, externally-induced transport through media of interest. Examples include transcellular or paracellular space, biological membranes, specific biologically and or disease-relevant barriers exemplified by hematoencephalic or peritumoral barriers, extracellular matrix, specific tissues, organs and/or blood/lymph vessels. Representative examples of shapes include but are not limited to helical (worm, screw-like), micro/nanopropellers, threads and/or ribbon-like, smooth, etched-surface sphere/spheroids, particles with or without one/multiple external appendage(s) as exemplified by cilia, flagellum/flagella, fin(s). Importantly, the particle design that combines elements responsible for securing electromagnetically-driven navigation (ferro/paramagnetic core), payload delivery (mesoporous materials) and ultrasound-triggered release (US-sensitive polymer) is unlikely to cause undesired performance interference (e.g., electromagnetically-triggered payload release or heating). It is technically feasible and could be accommodated via existing commercial equipment as exemplified by a Helmholtz magnetic coil set-up and common diagnostic ultrasound equipment. Moreover, due to both the active transport of the particles and expeditious target, tissue and/or organ delivery, release of the payload and retraction of the particles, the particle-(in vivo) system interaction is carefully controlled, limited and unlikely to trigger unwanted physiological effect(s) as exemplified by immunological, inflammatory and/or metabolic responses. In order to further minimize potential in vivo side effects that could be triggered by the US-induced polymer core rupture, a specific selection of biocompatible, non-toxic biodegradable polymers or a combination of thereof could be applied. Representative polymers include but are not limited to polyvinylalcohol (PVA), polyethyleneglycol (PEG), poly (N-2-hydroxypropyl) methacrylamide, poly(N-isopropyl)acrylamide, polylactic acid, chitosan, and polyglycolide.

In other embodiments of this invention, US-mediated heating of the particle is carefully controlled via i) US wave intensity; ii) US exposure time; and iii) particle nature including composition (content of ferro/paramagnetic, mesoporous components), size, shape and surface/coating. These embodiments i) maintain a proper balance between ferro/paramagnetic component sufficient to propel the particle via the external magnetic field and uncontrolled and/or rapid US-mediated heating; ii) control the heating rate by selecting a helical-shaped particle that is expected to acquire and transfer heat at slower rates than that a cylindrical particle of the same dimensions; and iii) treat particles with multi-layered biocompatible polymer coating to provide better control over heat absorption and dissipation.

In further embodiments, particles may feature both (a) multiple payloads (small molecules, biologics, antibodies, antisense oligonucleotides, RNAs, aptamers, peptide/peptoids, viruses); and (b) differential US-sensitive chemistries specifically responsive to a narrow US wave envelope e.g., 1 MHz vs 12 MHz. Specifically, these particles can be prepared via a variety of physical (multi-layered coating, prefabrication, shape-memory, etching) or chemical (e.g., polymer length, (co)polymerization, application of diverse polymers as exemplified by PDMS vs THMRA vs polymethyl metacrylate (PMMA), complexing, gating molecules with response optimized to a specific US intensity) techniques. Application of these customized particles in a single treatment or in separate installments further enhances efficacy/safety ratio to treat a patient-specific condition (e.g., a particular organ tumor with unique genetic signature/profile). Specifically, one could (a) deliver a cancer (e.g., gene, protein, pathway, network)-specific payload(s); (b) achieve a carefully controlled longitudinal or spatial release; and c) accelerate-slow down-stop the release of a payload by varying the intensity/timing of ultrasound and/or particle composition, namely ferromagnetic, mesoporous components and coating/gating techniques.

In a specific embodiment of the invention, i) US-mediated acoustic cavitation protocol (targeted frequencies of 20-100 kHz); or ii) direct US-mediated rupture of the polymer coat (targeted frequencies of 0.5-12 MHz); is used to induce payload release from the described composite ferro/paramagnetic mesoporous particles. In a representative synthetic protocol, composite particles of the specified geometry (e.g., micro/nano screws), size (e.g., 100 nm-1,000 μm) synthesized as described above (e.g., via GLAD) and containing ferro/paramagnetic core (e.g., Fe₃O₄, Ni, Co), mesoporous material (e.g., ZrO exhibiting pores of >10 nm) are treated with a saturated solution of a targeted payload (e.g., doxorubicin, paclitaxel or cisplatin) in organic solvent (e.g., acetonitrile, dimethylformamide, dioxane), water, buffer or a mixture of miscible organic solvent and water with gentle stirring and under inert atmosphere (e.g., Ar or N₂). The resulting suspension is filtered off, washed with acetonitrile and/or water/acetonitrile (ca. 50/50%) followed by dry acetonitrile and ether. The collected particles are resuspended in a 1-25% coating solution of a specific polymer (e.g., polystyrene) in organic solvent (e.g., toluene, dioxane, acetonitrile) for 2 hours with gentle stirring followed by filtration and gradual drying in a stream of dry air or Na gas to produce targeted composite particles featuring payload entrapped by the mesoporous component and sealed with a coat of polymer. The resulting particles are not expected to release the payload without specific US treatment. In a representative acoustic cavitation-mediated payload release protocol (US frequencies of 20-100 kHz), the particles are placed or navigated using magnetic field into a specific location followed by US treatment (5 min at 25° C., 20 kHz, 15 W/cm², constant or 5 sec ON/OFF pulse) to rupture the polymer coat and to release entrapped payload.

Optimization of payload release (the ratio of released vs total loaded payload per unit of time) can be achieved by altering particle nature or US treatment protocol. For example, to optimize particle nature one would vary: i) the polymer chemistry (e.g., changing polystyrene to PVA); ii) polymer MW; iii) polymer concentration in the coating solution (see experimental protocol above); iv) duration of coating step (e.g., 2 hours vs 4 hours); v) specific surface pretreatment or assisted coating (e.g., enhancement of porosity during GLAD, addition of specific chemical components as exemplified by Al₂O₃) to enhance coat adhesion and/or coat stability; or vi) apply additional coating steps with the same or distinct US sensitive polymer to increase thickness or stability of the resulting film (e.g., PVA followed by THPMA coating steps). To optimize US treatment protocol, one would modulate: i) frequency (e.g., 20 vs 50 kHz for acoustic cavitation protocol); ii) power (e.g., increase/decrease US source output from 15 to 25 W/cm²) and power dissipation; iii) duration of treatment (e.g., 30 sec vs 180 sec); iv) US aperture/focus to concentrate US energy on the smaller treatment volume; or v) apply constant or pulsing US. In a representative example, selection of a polymer coat (e.g., polystyrene or chitosan vs THPMA) yields frequency (e.g., 20 kHz vs 1 MHz) and/or protocol specific (e.g., acoustic cavitation vs direct polymer degradation) coat effect.

In summary, the embodiments described above provide actively navigated, tractable magnetic mesoporous composite nano-microparticles that deliver and release the targeted payload at precise location(s) via the US frequency-specific coat rupture at low vs high frequencies. The release of the payload can be tuned up to a specific US frequency as exemplified by either an acoustic cavitation protocol (targeted frequencies of 20-100 kHz) or direct US polymer effect (targeted frequencies of 0.5-12 MHz). Multiple specific factors including physical parameters of the US wave and particle chemistry are amenable to optimization to achieve selective, precise, safe and efficacious delivery of the payload to the target ex vivo or in vivo.

In some embodiments of this invention, the propelling component can be the same component that includes the functional material in it or on it. According to this aspect and in one embodiment, the propelling element comprises a feature that enables propulsion (e.g. a magnetic component) and a feature capable of containing the functional material. For example, with reference to FIG. 1.2, a ferromagnetic or paramagnetic composite rod can be a porous composite where the functional material is attached to the ferro/para-magnetic rod. Such component can assume any shape, form and size applicable to embodiments of this invention.

As indicated herein, the polymeric coating is optional. In embodiments of this invention, no coating is provided, and the device is constructed such that the functional material is attached to the structure/component in a way that keeps it intact as the device moves inside or along a tissue. When or where the functional material is needed, a remote trigger/stimulus is applied and the functional material (or any entity comprising the functional material) detaches from the structure/component and released to the targeted region where it is needed.

In other embodiments and for example but not only where the contact between the functional material and the structure is not very stable, the polymeric coating (shown for example in FIG. 1.3) provides protection for the functional material during the period where the device is moving toward the release target. The coating material is opened at the target to release the target material. The coating material provides extra control over the release process. The release process is controlled by gradual opening, immediate opening and/or by open/close operation of the coating to allow for enhanced release parameters.

In embodiments of this invention where stimuli is noted, the embodiment also refer to a single stimulus in some embodiments.

The design described in this embodiment support features 1-4 as defined above in the summary section, as the selection of the target frequency X directly defines the possible penetration depth, as well as enables individual control of several carriers in a single unit volume. Each carrier can be designed to have a different resonant frequency, thus allowing individual activation of a single carrier by a specific US signal.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A carrier device for implanting in a biological tissue for release of a functional material in said tissue or in another tissue, the carrier device comprising:
a structure comprising a propelling component;
a functional material attached directly or through a linker to said structure; and
optionally a coating, said coating at least partially covers said structure and at least partially covers said functional material attached to said structure.

2. The device of claim 1, wherein said propelling component is a magnetic component.

3. The device of claim 1, wherein said propelling component, said functional material, said coating or a combination thereof are responsive to external stimuli.

4. The device of claim 3, wherein said stimuli are selected from US, magnetic, electric, electromagnetic, electromagnetic radiation or a combination thereof.

5. The device of claim 4, wherein application of said stimuli to said propelling component propels said device.

6. The device of claim 3, wherein:
o said functional material detaches from said structure in response to said external stimuli; or
said coating ruptures or becomes perforated in response to said external stimuli; or
a combination thereof.

7. The device of claim 6, wherein said external stimuli is US.

8. The device of claim 1, wherein said structure is at least partially porous.

9. The device of claim 8, wherein the average pore size of said porous structure ranges between 10 nm-1000 nm.

10. The device of claim 1, wherein said functional material is an organic compound, a polymer, a composite or a combination thereof.

11. The device of claim 1, wherein said coating comprising a polymer, a composite or a combination thereof.

12. The device of claim 1, wherein said structure is a microstructure, a nanostructure or a combination thereof.

13. A system comprising:
the device of claim 1; and
a remote unit;
wherein said remote unit is configured to apply external stimuli to said device.

14. The system of claim 13, wherein said external stimuli comprises US.

15. The system of claim 13, wherein:
said functional material changes its shape or topology, or detaches from said structure in response to said external stimuli; or
said coating ruptures or becomes perforated in response to said external stimuli; or
a combination thereof.

16. A method for operating a device, said method comprising:
providing a carrier device comprising:
a structure comprising a propelling component;
a functional material attached directly or through a linker to said structure;
optionally a coating, said coating at least partially covers said structure and at least partially covers said functional material attached to said structure; and
applying external stimuli to said device.

17. The method of claim 16, wherein said coating, said functional material or a combination thereof are responsive to said external stimuli.

18. The method of claim 16, wherein said stimuli is US.

19. The method of claim 16, wherein:
said functional material detaches from said structure in response to said external stimuli; or
said coating ruptures or becomes perforated in response to said external stimuli; or
a combination thereof.

20. The method of claim 16, wherein said functional material is an organic compound, a polymer, a composite or a combination thereof.

21. The method of claim 16, wherein said coating comprising a polymer, a composite or a combination thereof.

22. The method of claim 16, wherein said structure is a microstructure, a nanostructure or a combination thereof.

23. The method of claim 16, wherein said propelling component comprises a magnetic component.

24. A method of producing the device of claim 1, said method comprising:
providing or constructing a propelling structure, said structure comprises a propelling element;
binding a functional material to said structure; and
optionally coating at least partially said structure or optionally coating at least partially said functional material or a combination thereof.

25. A method of treating a subject, said method comprises:
inserting the device of claim 1 into said subject;
applying external stimuli to said device.

26. The method of claim 25, wherein said inserting the device comprises inserting the device into a certain tissue within said subject.

27. The method of claim 25, wherein said external stimuli comprises:
magnetic/electric or electromagnetic stimuli to propel the device to a defined location within the subject; or
US stimuli to detach said functional material from said structure and/or to rupture or perforate said coating; or
a combination thereof.

28. The method of claim 27, wherein following application of said external stimuli, said functional material interacts with said tissue or with component(s) of/in said tissue.

29. The method of claim 28, wherein said interaction results in a therapeutic effect, a diagnostic effect or a combination thereof.

30. The method of claim 25, further comprising imaging the location of said device within said subject.

31. The method of claim 25, wherein said propelling component is a magnetic component.
